(19) United States
(12) Patent Application Publication
(10) Pub. No.: US 2008/0076721 A1
(43) Pub. Date: Mar. 27, 2008

(54) Use of branched chain fatty acid amino acid salts and compositions thereof

(76) Inventors: Sal Abraham, Easton, PA (US); Shengli Jiang, Taizhou (CN)

Correspondence Address: SAL ABRAHAM, 10901 TAMORON LANE, BOCA RATON, FL, 33498, US

(21) Appl. No.: 11/535,061

(22) Filed: Sep. 25, 2006

(51) Int. Cl. A61K 38/05; A61K 38/04; A61K 31/4172; A61K 31/407; A61K 31/401; A61K 31/205

(52) U.S. Cl. 514/18; 514/400; 514/419; 514/423; 514/554; 514/19

(57) ABSTRACT

The present invention relates to the use of naturally occurring methylated branched chain fatty acids, precursors, or derivatives such as but not limited to 3,7,11,15-tetramethyl-2-Hexadecen-1-ol (phytol), 3,7,11,15-tetramethylhexadec-2-enoic acid (phytenic acid), 3,7,11,15-tetramethylhexadecanoic (phytanic acid) or 2,6,10,14-tetramethylpentadecanoic (pristanic acid) as amino acid salts for cosmetic, dietary supplement, or pharmaceutical compositions, which are useful in increasing the variables associated with physical performance for the regulation of athletic function.

### BACKGROUND OF INVENTION

The present invention relates to the novel and unobvious use of methylated branched chain fatty acid amino acid salts. The unique combination of branched chain fatty acids and amino acids creates a superior ingredient, which enhances absorption and efficacy due to improved solubility and dual functionality. The following invention may be utilized as a cosmetic, dietary supplement, or pharmaceutical composition, which is useful in increasing type II muscle fiber for the regulation of athletic function.

The human diet contains phytol, a metabolite of the chlorophyll molecule. Phytol is metabolized to phytenic acid and phytanic acid. Intestinal absorption of phytol from dietary chlorophyll has been shown to be minimal. Dairy products and ruminant fats in the human diet are the major sources of phytanic acid. A normal diet contains 50 to 100 mg of phytanic acid per day (Steinberg. pp. 2351-2369, McGraw-Hill, N.Y. 1995). Phytanic acid may be elevated in patients with Refsum's disease, an inherited metabolic disorder characterized by an alpha-hydroxylase gene defect that prevents the conversion of phytanic acid to pristanic acid.

U.S. Pat. No. 6,784,207 by Fleuhmann et al describes a method for the treatment or prevention of preferably non-insulin dependent (NIDDM or so-called Type II) diabetes mellitus, or other conditions associated with impaired glucose tolerance such as obesity, and in particular to the use of phytanic acid derivatives for the said treatment and/or prevention. However, while this may be an improvement in the treatment and prevention of diabetes it does not demonstrate the use of branched chain fatty acids as amino acid salts for cosmetic, dietary supplement, or pharmaceutical compositions, which are useful in increasing type II muscle fiber for the regulation of athletic function.

U.S. patent application number 20040138181 by Fleuhmann Beet describes a method for the treatment or prevention of preferably non-insulin dependent (NIDDM or so-called Type II) diabetes mellitus, or other conditions associated with impaired glucose tolerance such as obesity, and in particular to the use of phytanic acid derivatives for the said treatment and/or prevention. A method of making a composition for the treatment or prevention of non-insulin dependent diabetes mellitus and related diseases comprising combining phytanic acid or derivatives thereof with a pharmaceutically acceptable additive or adjuvant, and a composition for the treatment or prevention of non-insulin dependent diabetes mellitus comprising phytanic acid or derivatives thereof are also provided. However, while this may be an improvement in the treatment and prevention of diabetes it does not demonstrate the use of branched chain fatty acids as amino acid salts for cosmetic, dietary supplement, or pharmaceutical compositions, which are useful in increasing type II muscle fiber for the regulation of athletic function.

### SUMMARY OF INVENTION

The present invention relates to the use of methylated branched chain fatty acids, precursors and derivatives such as but not limited to 3,7,11,15-tetramethyl-2-Hexadecen-1-ol (phytol), 3,7,11,15-tetramethylhexadec-2-enoic acid (phytenic acid), 3,7,11,15-tetramethylhexadecanoic acid (phytanic acid) or 2,6,10,14-tetramethylpentadecanoic (pristanic acid). The method comprises administering to humans an effective amount of a composition consisting of branched chain fatty acid amino acid salts such as but not limited to L-arginine phytanate, Creatine phytanate, and L-carnitine phytanate. These novel and unobvious combinations create branched chain fatty acid amino acid salts, which are molecularly attached by an ionic bond to form new highly bioavailable ingredients. Branched chain fatty acid amino acid salts are vastly superior to all other types of branched chain fatty acids. This molecular attachment creates a compound that is both lipophilic (fat loving) and hydrophilic (water loving). This dual solubility enhances absorption and bioavailability thus resulting in better retention and utilization by the body.

The problem of the present invention is to provide a superior, legal, safe, and efficacious alternative to performance and physique enhancing dietary supplements such as arachidonic acid (AA), (OEA) oleylethanolamide, and 3-thia substituted fatty acids. Arachidonic acid is thought to increase muscle mass due to its role the inflammation pathway; however, in vitro research has implicated arachidonic acid with prostate cancer cell growth and possible cardiovascular implications (Cancer Res. 66(3): 1427-33, 2006 and Carcinogenesis 22:701-7, 2001). The consumption of healthy fats such as omega-3 fatty acids attenuates the inflammation pathway thus negating arachidonic acid's possible role in muscle growth. OEA has been researched by Kadmus Pharmaceutical as a drug prior to being sold as supplement and therefore may not be legally sold as a dietary supplement. 3-Thia fatty acids, such as tetradecylthioacetic acid, developed and patented by Thia Medica AS, are questionable ingredients because it is not readily apparent whether these ingredients fit within the DSHEA section three definition of a dietary ingredient. According to the present invention these problems are solved by the use of branched chain fatty acid amino acid salts. There is extensive scientific literature regarding the health benefits of branched chain fatty acids in addition to the fact these ingredients occur in nature and thus would be considered a botanical constituent or dietary substance not sold or researched as drugs prior to being sold as a dietary supplement, which would be categorized as a legal dietary ingredient or supplement.

L-arginine phytanate with a molecular formula of C₂₅H₅₁N₄O₄ and a molecule weight of 471.7 solves the problem of the present invention. Without being bound to any theory, the branched chain fatty acid amino acid salt produces stable and soluble amino acid salts. Once the intact compound enters the blood stream, it exerts a synergistic effect since both the phytanic acid and amino acid contribute to the promotion of lean tissue, fat loss, endurance, strength, and recovery by different and similar mechanisms. 3,7,11,15-tetramethylhexadecanoic (phytanic acid) is a natural ligand for PPAR alpha and to a lesser degree beta/delta and gamma. (Biochim Biophys Acta. 1521(1-3): 97-106, Oct 31, 2001). Phytanic acid is thought to decrease body fat and increase insulin sensitivity due to its high affinity for the PPAR alpha-receptor. Phytanic acid also possesses the ability to increase nitric oxide production via increased nitric oxide synthase (J. Biol. Chem., 277(51), 49319-49325,Dec. 20, 2002). L-arginine is a precursor to nitric oxide production and thus works synergistically with phytanic acid. The use of branched chain fatty acid amino acid salts such as L-arginine phytanate possesses the ability to increase type 11 muscle fiber and strength when combined with a high protein diet and resistance training program due to its wide range of nuclear receptor PPAR activation and dual nitric oxide production. To the best of my knowledge, I purpose for the first time the use of branched chain fatty acid amino acid salts as cosmetic, dietary supplement, or pharmaceutical compositions. It should be understood that this invention is not construed as limited in scope by the details contained therein, as it is apparent to those skilled in the art that modifications in materials and methods can be made without deviating from the scope of the invention.

### DETAILED DESCRIPTION

Branched chain fatty acids are common components of the lipids of bacteria and animals. The fatty acyl chain is usually saturated and the branch is a methyl-group. Phytol is an aliphatic alcohol moiety found in chlorophyll, which metabolizes to a number of isoprenoid 3-methyl fatty acids. The biosynthetic pathway of phytol produces phytenic acid, phytanic acid, phytanoyl-Coa, 2-hydroxyphytanoyl-Coa, pristanal, and pristanic acid respectively. The shorter chain isoprenoid fatty acids are formed by alpha and/or beta-oxidation reactions. The methyl groups of natural phytanic acid are expected to have the D-configuration while phytanic acid synthesized via chemical hydrogenation of phytol contain a racemic mixture of D and L.

Peroxisome proliferator-activated receptors (PPARs) are members of the nuclear hormone receptor super family of ligand-activated transcription factors that are related to retinoid, steroid and thyroid hormone receptors. The PPAR sub types play an important role in many cellular functions including lipid metabolism, cell proliferation, differentiation, adipogenesis and inflammatory signaling. The PPAR sub types have been found to interact with a number of endogenous lipids and drugs for the treatment of human metabolic diseases. There are three distinct PPAR subtypes that are the products of different genes and are commonly designated PPAR alpha, PPAR beta or delta, and PPAR gamma. PPARs regulate gene expression by complexing with a heterodimeric partner RXR (retinoid X receptors) and subsequent binding to specific response elements (PPREs) in the promoter regions of target genes.

Ronald Evans has demonstrated that the activation of the PPAR beta/delta receptor with site-specific synthetic drugs can influence aerobic endurance in animals. (Wang Y X, et al. PLoS Biol 2(10): e294, 2004) Aerobic endurance training can cause a transformation of muscle fiber due to years of training. Aerobic endurance training causes an increase in type I muscle fiber. Experiments with synthetic selective PPAR beta/delta agonists have been shown to possess the ability to replicate years of aerobic endurance via gene transcription in a short time period. In this experiment, the average untrained mouse was given a synthetic selective PPAR beta/delta agonist, which enabled the untrained mice to run the same distance as the trained marathon mice. Activation of this receptor also caused similar physiological changes that take place in aerobically trained mice.

The chemical terms branched chain fatty acid amino acid salt, precursors, and derivatives include such as but not limited to L-arginine phytanate or 3,7,11,15-tetramethylhexadecanoic L-arginine salt. It can also refer to L-glutamine phytenate. Possible alternative amino acid salts of phytanic acid include the use of all essential and non-essestial L-form, D-form, and DL-form amino acids and dipeptides such as Glycylarginine phytanate, Glycylglutamine phytanate, Alanylarginine phytanate, and Alanylglutamine phytanate. This invention concerns a branched chain fatty acid amino acid salt in various ratios preferably a 1:1 ratio and all previously mentioned alternatives. The previous examples of various ratios and amino acid salts are presented by way of illustration only. It should be understood that this invention is not construed as limited in scope by the details contained therein, as it is apparent to those skilled in the art that modifications in materials and methods can be made without deviating from the scope of the invention.

The addition of different amino acids to phytanic acid can alter its functionality. Creatine phytanate emphasizes strength, endurance and muscle size while L-carnitine phytanate emphasizes muscle hardening. 3,7,11,15-tetramethylhexadecanoic-L-arginine salt meets the DSHEA section three definition of a dietary supplement. Phytanic acid has been described as a ligand for both RXR, PPAR alpha, PPAR beta or delta, and PPAR gamma. PPAR forms permissive heterodimers with RXR, meaning that either partner can regulate the transcriptional activity by interacting with its own ligand. Co-treatment of the cells with ligands for PPAR as well as RXR results in an additive effect.

The branch chain fatty acid amino acid salt promotes lean type II muscle fiber unlike regular branched chain fatty acids. Thus, the said compound can be given to humans either in conjunction with or without a high protein diet (1.25 to 1.8 grams protein/ kilogram of body weight) and proper anaerobic training program in order to increase the variables associated with athletic function for enhancing physical performance. Therefore, these compounds represent a substantial improvement in the efficacy and safety of fatty acid supplementation, which may now be legally utilized with mammals.

After an extensive review of the scientific literature regarding the novel, unobvious, and superior use of branched chain fatty acid amino acid salts it then became the focus of this invention that branched chain amino acid salts could be administrated as an effective means of regulating athletic function in experienced weight trained individuals. The oral daily doses can be between 0.1 to 40,000 mg., but preferably 0.1 to 10,000 mg/day, but even more preferably 0.1 to 5000 mg/day. In addition to peroral use, several other routes such as transdermal, sublingual, intranasal, or parenteral, can effectively administer branched chain fatty acid amino acid salts. Other dosage forms and applications include capsules, tablets, caplets, liquids, powders or functional food products.

The following example illustrates a method of use for L-arginine phytanate. The following examples should not be considered as limitations of the present invention.

### EXAMPLE 1

### L-Arginine Phytanate Method of Use

In this example a 36-year-old experienced weight trained male orally consumes 3 grams of L-arginine phytanate post workout for 4 weeks as a beverage, consumes no supplements for 4 weeks, then orally consumes phytanic acid in the same manner post workout for 4 weeks. The L-arginine phytanate is incorporated into free powder mixture of flavoring agents and palatinose, which is then combined with water. Once in the plasma L-arginine phytanate then exerts a synergistic lean tissue promoting effect due to the anabolic actions of phytanic acid and L-arginine. Phytanic acid activates PPAR alpha, beta/delta, gamma, and increases nitric oxide synthase thus increasing nitric oxide production. This wide range of PPAR receptor activation and nitric oxide release combined with a high protein diet and anaerobic training program, enhances muscle building and fat loss. The L-arginine synergistically promotes protein synthesis and nitric oxide release. These physiological functions lead to increases in lean body mass, strength, endurance, recovery and loss of body fat. The test subject reported a weight gain of 5 lbs., a 25-pound increase in 1 repetition maximum (RM) bench press, and 6-repetition increase in initial 70 percent maximum bench press at the end of the L-arginine phytanate 4-week period as compared to baseline values. These increases contribute to the regulation of athletic function and thus lead to enhanced physical performance. The test subject then abstained from all dietary supplements for 4 weeks, which resulted in most variables returning to baseline. During the final phase of the experiment, the test subject reported no increases in body weight, 1 repetition maximum bench press, or 70 percent maximum bench press. However, the individual reported a decrease in body weight at the end of the phytanic acid 4-week period.

The foregoing descriptions of the invention are for illustration only. Modifications not included in the description, which are obvious to those skilled in the art, are intended to be included in the scope of the following claims.

What is claimed is:

1. A method for providing a branched chain fatty acid or amino acid to a mammal, comprising: receiving a branched chain fatty acid amino acid salt by the mammal, wherein the branched chain fatty acid amino acid salt is suitable for being modified by the mammal to form a branched chain fatty acid or an amino acid.

2. The method as described in claim 1, wherein said branched chain fatty acid amino acid salt is suitable for peroral, transdermal, sublingual, intranasal, or parenteral administration.

3. The method as described in claim 1, wherein said branched chain fatty acid amino acid salt is suitable in a daily dose of 0.1 to 40,000 mg per day.

4. The method as described in claim 1, wherein said branched chain fatty acid amino acid salt is received by the mammal, the branched chain fatty acid amino acid salt is modified by the mammal into a branched chain fatty acid and an amino acid.

5. The method as described in claim 1, wherein said branched chain fatty acid is selected from the group consisting of 3,7,11,15-tetramethylhexadec-2-enoic acid (phytenic acid), 3,7,11,15-tetramethylhexadecanoic acid (phytanic acid) or 2,6,10,14-tetramethylpentadecanoic acid (pristanic acid) or combinations thereof or L-form, D-form, or DL-form stereoisomers of said branched chain fatty acids thereof or esters, salts, ethers, analogs, derivatives or precursors of said branched chain fatty acids thereof.

6. The method as described in claim 1, wherein said amino acid is selected from the group consisting of alanine, arginine, asparagine, aspartic acid, carnosine, carnitine, citrulline, creatine, cysteine, cystine, glutamine, glutamic acid, glycine, histamine, histidine, hydroxyproline, isoleucine, leucine, lysine, methionine, ornithine, phenylalanine, phenylglycine, proline, pyroglutamic acid, pyrrolysine, serine, theanine, threonine, tryptophan, tyramine, tyrosine, valine, or combinations thereof, or dipeptides or tripeptides formed by any combination of said amino acids thereof or L-form, D-form, or DL-form stereoisomers of said amino acids thereof or esters, salts, ethers, analogs, or derivatives of said amino acids thereof.

7. The method as described in claim 1, wherein the said mammal is selected from the group consisting of a human or livestock.

8. A method for supplementing the diet of a mammal, comprising administering as part of the diet an effective amount of a branched chain fatty acid and an effective amount of an amino acid.

9. The method as described in claim 8, wherein said branched chain fatty acid or amino acid is suitable for peroral, transdermal, sublingual, intranasal, or parenteral administration.

10. The method as described in claim 8, wherein said branched chain fatty acid or amino acid is suitable in a daily dose of 0.1 to 40,000 mg per day.

11. The method as described in claim 8, wherein said branched chain fatty acid is selected from the group consisting of 3,7,11,15-tetramethylhexadec-2-enoic acid (phytenic acid), 3,7,11,15-tetramethylhexadecanoic acid (phytanic acid) or 2,6,10,14-tetramethylpentadecanoic acid (pristanic acid) or combinations thereof or L-form, D-form, or DL-form stereoisomers of said branched chain fatty acids thereof or esters, salts, ethers, analogs, derivatives or precursors of said branched chain fatty acids thereof.

12. The method as described in claim 8, wherein said amino acid is selected from the group consisting of alanine, arginine, asparagine, aspartic acid, carnosine, carnitine, citrulline, creatine, cysteine, cystine, glutamine, glutamic acid, glycine, histamine, histidine, hydroxyproline, isoleucine, leucine, lysine, methionine, ornithine, phenylalanine, phenylglycine, proline, pyroglutamic acid, pyrrolysine, serine, theanine, threonine, tryptophan, tyramine, tyrosine, valine, or combinations thereof, or dipeptides or tripeptides formed by any combination of said amino acids thereof or L-form, D-form, or DL-form stereoisomers of said amino acids thereof or esters, salts, ethers, analogs, or derivatives of said amino acids thereof.

13. The method as described in claim 8, wherein the said mammal is selected from the group consisting of a human or livestock.

14. A method of increasing type II muscle fiber in a mammal in need thereof by administration to said mammal an effective amount of a branched chain fatty acid amino acid salt.

15. The method as described in claim 14, wherein said branched chain fatty acid amino acid salt is suitable for peroral, transdermal, sublingual, intranasal, or parenteral administration.

16. The method as described in claim 14, wherein said branched chain fatty acid amino acid salt is suitable in a daily dose of 0.1 to 40,000 mg per day.

17. The method as described in claim 14, wherein the branched chain fatty acid is selected from the group consisting of 3,7,11,15-tetramethylhexadec-2-enoic acid (phytenic acid), 3,7,11,15-tetramethylhexadecanoic acid (phytanic acid) or 2,6,10,14-tetramethylpentadecanoic acid (pristanic acid) or combinations thereof or L-form, D-form, or DL-form stereoisomers of said branched chain fatty acids thereof or esters, salts, ethers, analogs, derivatives or precursors of said branched chain fatty acids thereof.

18. The method as described in claim 14, wherein the amino acid is selected from the group consisting of alanine, arginine, asparagine, aspartic acid, carnosine, carnitine, citrulline, creatine, cysteine, cystine, glutamine, glutamic acid, glycine, histamine, histidine, hydroxyproline, isoleucine, leucine, lysine, methionine, ornithine, phenylalanine, phenylglycine, proline, pyroglutamic acid, pyrrolysine, serine, theanine, threonine, tryptophan, tyramine, tyrosine, valine, or combinations thereof, or dipeptides or tripeptides formed by any combination of said amino acids thereof or L-form, D-form, or DL-form stereoisomers of said amino acids thereof or esters, salts, ethers, analogs, or derivatives of said amino acids thereof.

19. The method as described in claim 14, wherein said mammal is selected from the group consisting of a human or livestock.
